(19) United States
(12) Patent Application Publication
(10) Pub. No.: US 2011/0172609 A1
(43) Pub. Date: Jul. 14, 2011

(54) MICRONEEDLE COMPONENT ASSEMBLY FOR DRUG DELIVERY DEVICE

(75) Inventors: Benjamin J. Moga, Madison, WI (US); Kent Chase, Sun Prairie, WI (US); Garrick D.S. Smith, Madison, WI (US)

(73) Assignees: Ratio, Inc.; FluGen, Inc.

(21) Appl. No.: 12/684,823

(22) Filed: Jan. 8, 2010

(51) Int. Cl. A61M 5/32; B23P 11/00

(52) U.S. Cl. 604/272; 29/428

(57) ABSTRACT

A device for delivering a drug to a subject is provided. The device includes a drug reservoir, a conduit coupled to the drug reservoir and a microneedle component. The microneedle component includes a body, an engagement structure coupling the microneedle component to the conduit, a hollow microneedle extending from the body, and a handling feature located on the body. The microneedle component is configured to be releasably coupled to an assembly tool via the handling feature during assembly of the device.

### BACKGROUND

The present invention relates generally to the field of drug delivery devices. The present invention relates specifically to an active transdermal drug delivery device including a microneedle component and a microneedle component assembly.

An active agent or drug (e.g., pharmaceuticals, vaccines, hormones, nutrients, etc.) may be administered to a patient through various means. For example, a drug may be ingested, inhaled, injected, delivered intravenously, etc. In some applications, a drug may be administered transdermally. In some transdermal applications, such as transdermal nicotine or birth control patches, a drug is absorbed through the skin. Passive transdermal patches often include an absorbent layer or membrane that is placed on the outer layer of the skin. The membrane typically contains a dose of a drug that is allowed to be absorbed through the skin to deliver the substance to the patient. Typically, only drugs that are readily absorbed through the outer layer of the skin may be delivered with such devices.

Other drug delivery devices are configured to provide for increased skin permeability to the delivered drugs. For example, some devices use a structure, such as one or more microneedles, to facilitate transfer of the drug into the skin. Solid microneedles may be coated with a dry drug substance. The puncture of the skin by the solid microneedles increases permeability of the skin allowing for absorption of the drug substance. Hollow microneedles may be used to provide a fluid channel for drug delivery below the outer layer of the skin. Other active transdermal devices utilize other mechanisms (e.g., iontophoresis, sonophoresis, etc.) to increase skin permeability to facilitate drug delivery.

### SUMMARY

One embodiment of the invention relates to a device for delivering a drug to a subject. The device includes a drug reservoir, a conduit coupled to the drug reservoir and a microneedle component. The microneedle component includes a body, an engagement structure coupling the microneedle component to the conduit, a hollow microneedle extending from the body, and a handling feature located on the body. The microneedle component is configured to be releasably coupled to an assembly tool via the handling feature during assembly of the device.

Another embodiment of the invention relates to microneedle component of a drug delivery device. The microneedle component includes a bottom wall having a lower surface, a sidewall coupled to the bottom wall and a microneedle extending from the lower surface of the bottom wall. The microneedle component also includes a robotic handling feature formed in the lower surface of the bottom wall that is configured to be releasably coupled to a robotic assembly tool during assembly of the drug delivery device.

Another embodiment of the invention relates to a method of manufacturing a drug delivery device. The method includes providing a microneedle component having a robotic handling feature, providing a drug reservoir and providing a conduit coupled to the drug reservoir. The method also includes coupling the microneedle component to a robotic assembly device via engagement between the robotic handling feature and the robotic assembly device and coupling the microneedle component to the conduit with the robotic assembly device.

Alternative exemplary embodiments relate to other features and combinations of features as may be generally recited in the claims

### BRIEF DESCRIPTION OF THE FIGURES

This application will become more fully understood from the following detailed description, taken in conjunction with the accompanying figures, wherein like reference numerals refer to like elements in which:

FIG. 1 is a perspective view of a drug delivery device assembly having a cover and a protective membrane according to an exemplary embodiment;

FIG. 2 is a perspective view of a drug delivery device according to an exemplary embodiment after both the cover and protective membrane have been removed;

FIG. 3 is a exploded perspective view of a drug delivery device assembly according to an exemplary embodiment;

FIG. 4 is a exploded perspective view of a drug delivery device showing various components mounted within the device housing according to an exemplary embodiment;

FIG. 5 is a exploded perspective view of a drug delivery device showing various components removed from the device housing according to an exemplary embodiment;

FIG. 6 is a perspective sectional view showing a drug delivery device prior to activation according to an exemplary embodiment;

FIG. 7 is a perspective sectional view showing a drug delivery device following activation according to an exemplary embodiment;

FIG. 8 is a side sectional view showing a drug delivery device following activation according to an exemplary embodiment;

FIG. 9 is a side sectional view showing a drug delivery device following delivery of a drug according to an exemplary embodiment;

FIG. 10 is a exploded view showing a microneedle component assembly for a drug delivery device according to an exemplary embodiment;

FIG. 11 is a perspective view of a microneedle component according to an exemplary embodiment;

FIG. 12 is a top view of a microneedle component according to an exemplary embodiment;

FIG. 13 is a bottom view of a microneedle component according to an exemplary embodiment;

FIG. 14 is a perspective view of a seal component according to an exemplary embodiment;

FIG. 15 is a bottom view of a microneedle attachment portion according to an exemplary embodiment;

FIG. 16 is a perspective view showing a microneedle component assembly for a drug delivery device according to an exemplary embodiment;

FIG. 17 is a sectional view shown a microneedle component assembly fro a drug delivery device according to an exemplary embodiment; and

FIG. 18 is a flow diagram showing an assembly process for a microneedle drug delivery device according to an exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring generally to the figures, a substance delivery device assembly is shown according to various exemplary embodiments. The delivery device assembly includes various packaging and/or protective elements that provide for protection during storage and transportation. The assembly also includes a substance delivery device that is placed in contact with the skin of a subject (e.g., a human or animal, etc.) prior to delivery of the substance to the subject. After the device is affixed to the skin of the subject, the device is activated in order to deliver the substance to the subject. Following delivery of the substance, the device is removed from the skin.

The delivery device described herein may be utilized to deliver any substance that may be desired. In one embodiment, the substance to be delivered is a drug, and the delivery device is a drug delivery device configured to deliver the drug to a subject. As used herein the term “drug” is intended to include any substance delivered to a subject for any therapeutic, preventative or medicinal purpose (e.g., vaccines, pharmaceuticals, nutrients, nutraceuticals, etc.). In one such embodiment, the drug delivery device is a vaccine delivery device configured to deliver a dose of vaccine to a subject. In one embodiment, the delivery device is configured to deliver a flu vaccine. The embodiments discussed herein relate primarily to a device configured to deliver a substance intradermally. In other embodiments, the device may be configured to deliver a substance transdermally or may be configured to deliver drugs directly to an organ other than the skin.

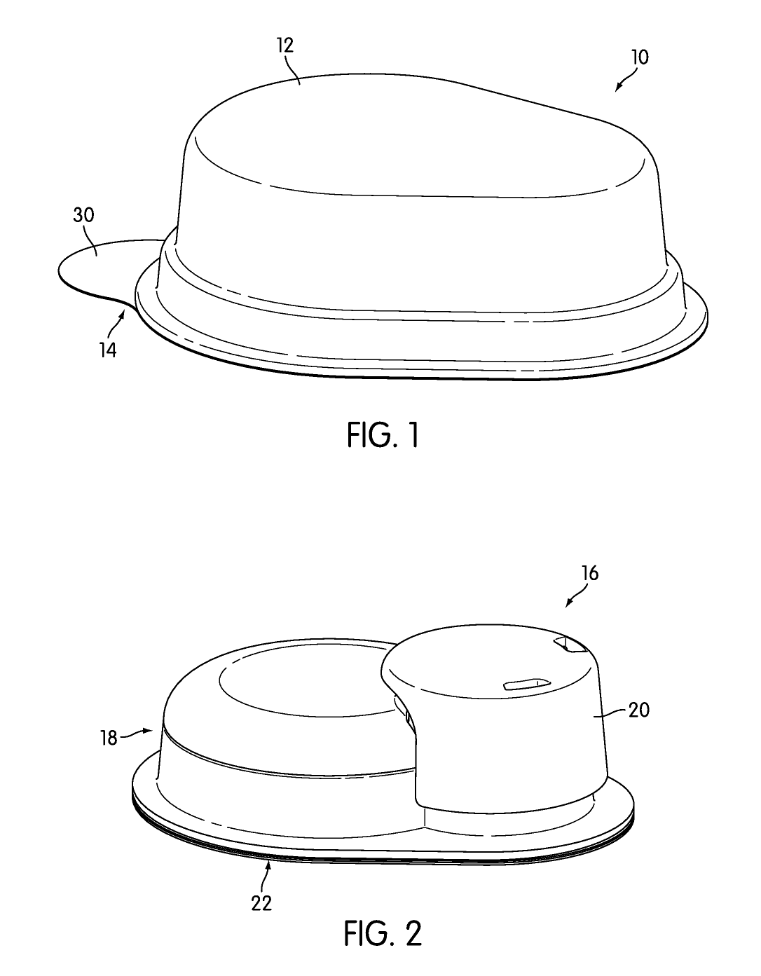

Referring to FIG. 1, drug delivery device assembly 10 is depicted according to an exemplary embodiment. Drug delivery device assembly 10 includes an outer protective cover 12 and a protective membrane or barrier 14 that provides a sterile seal for drug delivery device assembly 10. As shown in FIG. 1, drug delivery device assembly 10 is shown with cover 12 and protective barrier 14 in an assembled configuration. Generally, cover 12 and protective barrier 14 protect various components of drug delivery device 16 during storage and transport prior to use by the end user. In various embodiments, cover 12 may be made of a relatively rigid material (e.g., plastic, metal, cardboard, etc.) suitable to protect other components of drug delivery device assembly 10 during storage or shipment. As shown, cover 12 is made from a non-transparent material. However, in other embodiments cover 12 is a transparent or semi-transparent material.

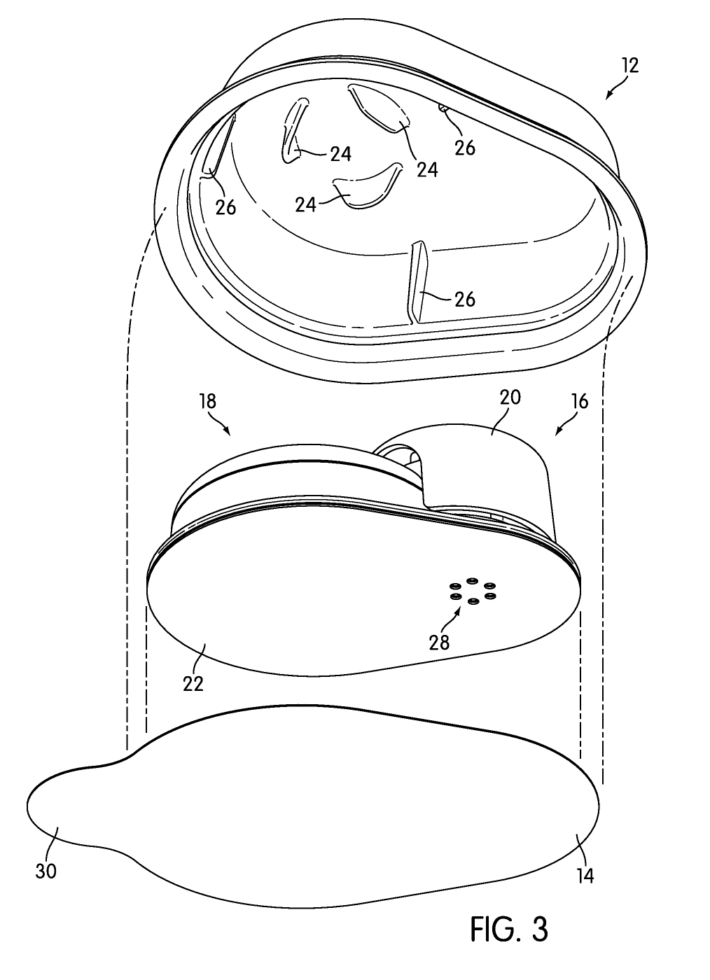

As shown in FIG. 2 and FIG. 3, the drug delivery device assembly includes delivery device 16. Delivery device 16 includes a housing 18, an activation control, shown as, but not limited to, button 20, and an attachment element, shown as, but not limited to, adhesive layer 22. Adhesive layer 22 includes one or more holes 28 (see FIG. 3). Holes 28 provide a passageway for one or more hollow drug delivery microneedles as discussed in more detail below. During storage and transport, cover 12 is mounted to housing 18 of delivery device 16 such that delivery device 16 is received within cover 12. In the embodiment shown, cover 12 includes three projections or tabs 24 extending from the inner surface of the top wall of cover 12 and three projections or tabs 26 extending from the inner surface of the sidewall of cover 12. When cover 12 is mounted to delivery device 16, tabs 24 and 26 contact the outer surface of housing 18 such that delivery device 16 is positioned properly and held within cover 12. Protective barrier 14 is attached to the lower portion of cover 12 covering adhesive layer 22 and holes 28 during storage and shipment. Together, cover 12 and protective barrier 14 act to provide a sterile and hermetically sealed packaging for delivery device 16.

Referring to FIG. 3, to use delivery device 16 to deliver a drug to a subject, protective barrier 14 is removed exposing adhesive layer 22. In the embodiment shown, protective barrier 14 includes a tab 30 that facilitates griping of protective barrier 14 during removal. Once adhesive layer 22 is exposed, delivery device 16 is placed on the skin. Adhesive layer 22 is made from an adhesive material that forms a nonpermanent bond with the skin of sufficient strength to hold delivery device 16 in place on the skin of the subject during use. Cover 12 is released from delivery device 16 exposing housing 18 and button 20 by squeezing the sides of cover 12. With delivery device 16 adhered to the skin of the subject, button 20 is pressed to trigger delivery of the drug to the patient. When delivery of the drug is complete, delivery device 16 may be detached from the skin of the subject by applying sufficient force to overcome the grip generated by adhesive layer 22.

In one embodiment, delivery device 16 is sized to be conveniently wearable by the user during drug delivery. In one embodiment, the length of delivery device 16 along the device's long axis is 53.3 mm, the length of delivery device 16 along the device's short axis (at its widest dimension) is 48 mm, and the height of delivery device 16 at button 20 following activation is 14.7 mm. However, in other embodiments other dimensions are suitable for a wearable drug delivery device. For example, in another embodiment, the length of delivery device 16 along the device's long axis is between 40 mm and 80 mm, the length of delivery device 16 along the device's short axis (at its widest dimension) is between 30 mm and 60 mm, and the height of delivery device 16 at button 20 following activation is between 5 mm and 30 mm. In another embodiment, the length of delivery device 16 along the device's long axis is between 50 mm and 55 mm, the length of delivery device 16 along the device's short axis (at its widest dimension) is between 45 mm and 50 mm, and the height of delivery device 16 at button 20 following activation is between 10 mm and 20 mm.

While in the embodiments shown the attachment element is shown as, but not limited to, adhesive layer 22, other attachment elements may be used. For example, in one embodiment, delivery device 16 may be attached via an elastic strap. In another embodiment, delivery device 16 may not include an attachment element and may be manually held in place during delivery of the drug. Further, while the activation control is shown as button 20, the activation control may be a switch, trigger, or other similar element, or may be more than one button, switch, trigger, etc., that allows the user to trigger delivery of the drug.

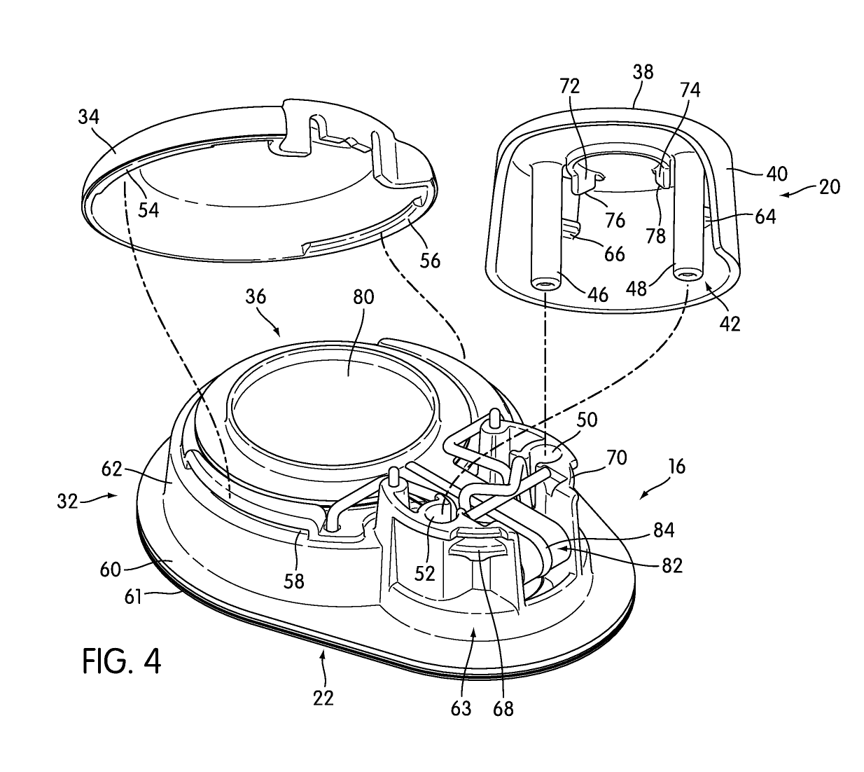

Referring to FIG. 4, housing 18 of delivery device 16 includes a base portion 32 and a reservoir cover 34. Base portion 32 includes a flange 60, a bottom tensile member, shown as bottom wall 61, a first support portion 62 and a second support portion 63. In the embodiment shown, bottom wall 61 is a rigid wall that is positioned below flange 60. As shown in FIG. 4, the outer surface of first support portion 62 is generally cylindrically shaped and extends upward from flange 60. Second support portion 63 is generally cylindrically shaped and extends upward from flange 60 to a height above first support portion 62. As shown in FIG. 4, delivery device 16 includes a substance delivery assembly 36 mounted within base portion 32 of housing 18.

Reservoir cover 34 includes a pair of tabs 54 and 56 that each extend inwardly from a portion of the inner edge of cover 34. Base portion 32 includes a recess 58 and second recess similar to recess 58 on the opposite side of base portion 32. As shown in FIG. 4, both recess 58 and the opposing recess are formed in the upper peripheral edge of the outer surface of first support portion 62. When reservoir cover 34 is mounted to base portion 32, tab 54 is received within recess 58 and tab 56 is received within the similar recess on the other side of base portion 32 to hold cover 34 to base portion 32.

As shown in FIG. 4, button 20 includes a top wall 38. Button 20 also includes a sidewall or skirt 40 that extends from a portion of the peripheral edge of top wall 38 such that skirt 40 defines an open segment 42. Button 20 is shaped to receive the generally cylindrical shaped second support portion 63 of base portion 32. Button 20 includes a first mounting post 46 and a second mounting post 48 both extending in a generally perpendicular direction from the lower surface of top wall 38. Second support portion 63 includes a first channel 50 and a second channel 52. Mounting posts 46 and 48 are slidably received within channels 50 and 52, respectively, when button 20 is mounted to second support portion 63. Mounting posts 46 and 48 and channels 50 and 52 act as a vertical movement guide for button 20 to help ensure that button 20 moves in a generally downward vertical direction in response to a downward force applied to top wall 38 during activation of delivery device 16. Precise downward movement of button 20 ensures button 20 interacts as intended with the necessary components of substance delivery assembly 36 during activation.

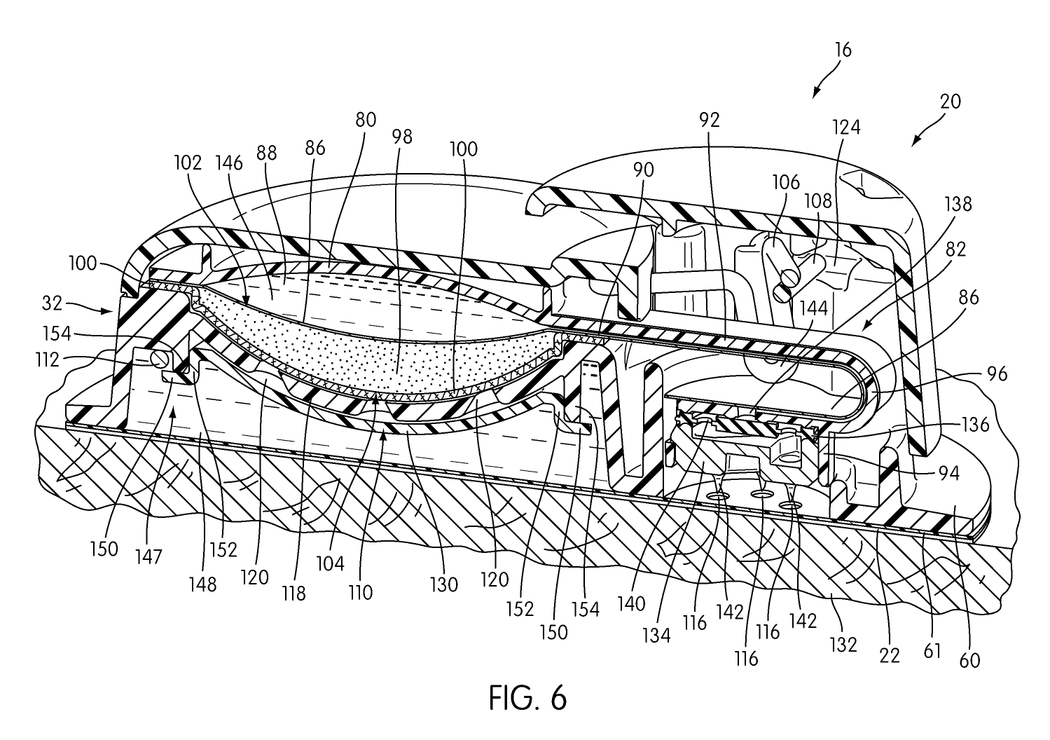

Button 20 also includes a first support ledge 64 and a second support ledge 66 both extending generally perpendicular to the inner surface of sidewall 40. The outer surface of second support portion 63 includes a first button support surface 68 and second button support surface 70. When button 20 is mounted to second support portion 63, first support ledge 64 engages and is supported by first button support surface 68 and second support ledge 66 engages and is supported by second button support surface 70. The engagement between ledge 64 and surface 68 and between ledge 66 and surface 70 supports button 20 in the pre-activation position (shown for example in FIG. 6). Button 20 also includes a first latch engagement element 72 and a second latch engagement element 74 both extending in a generally perpendicular direction from the lower surface of top wall 38. First latch engagement element 72 includes an angled engagement surface 76 and second latch engagement element 74 includes an angled engagement surface 78.

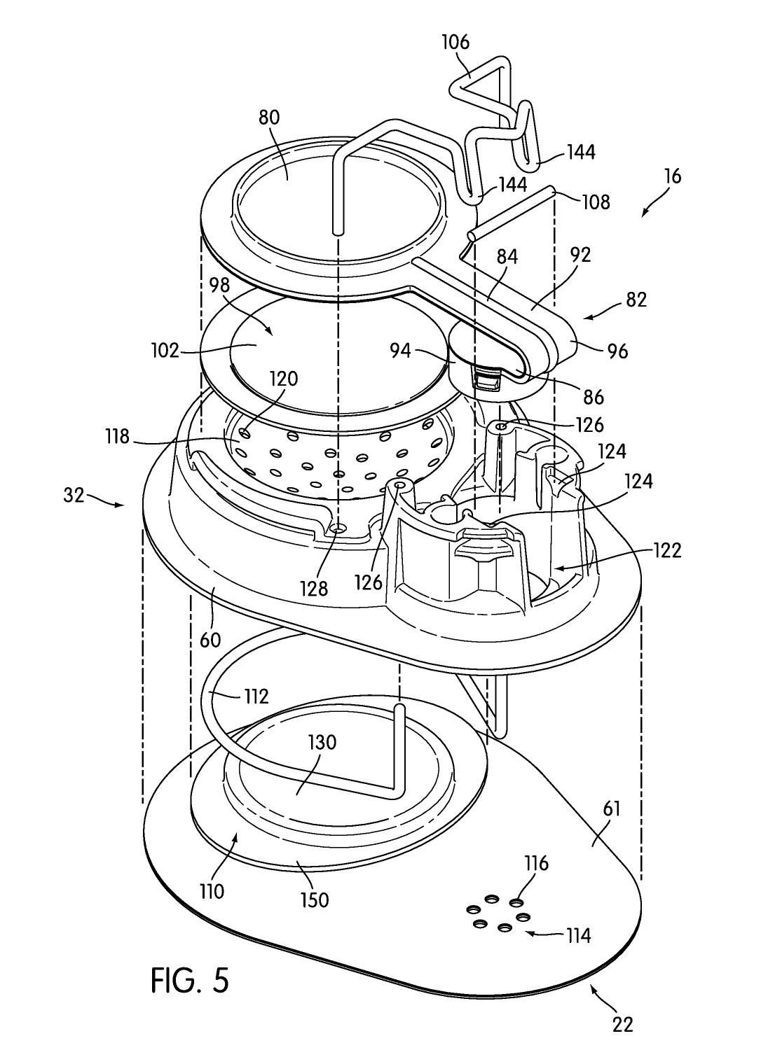

Referring to FIG. 4 and FIG. 5, substance delivery assembly 36 includes a drug reservoir base 80 and drug channel arm 82. The lower surface of drug channel arm 82 includes a depression or groove 84 that extends from reservoir base 80 along the length of drug channel arm 82. As shown in FIG. 4 and FIG. 5, groove 84 appears as a rib protruding from the upper surface of drug channel arm 82. Substance delivery assembly 36 further includes a flexible barrier film 86 adhered to the inner surfaces of both drug reservoir base 80 and drug channel arm 82. Barrier film 86 is adhered to form a fluid tight seal or a hermetic seal with drug reservoir base 80 and channel arm 82. In this arrangement (shown best in FIGS. 6-9), the inner surface of drug reservoir base 80 and the inner surface of barrier film 86 form a drug reservoir 88, and the inner surface of groove 84 and the inner surface of barrier film 86 form a fluid channel, shown as, but not limited to, drug channel 90. In this embodiment, drug channel arm 82 acts as a conduit to allow fluid to flow from drug reservoir 88. As shown, drug channel arm 82 includes a first portion 92 extending from drug reservoir base 80, a microneedle attachment portion, shown as, but not limited to, cup portion 94, and a generally U-shaped portion 96 joining the first portion 92 to the cup portion 94. In the embodiment shown, drug reservoir base 80 and drug channel arm 82 are made from an integral piece of polypropylene. However, in other embodiments, drug reservoir base 80 and drug channel arm 82 may be separate pieces joined together and may be made from other plastics or other materials.

Substance delivery assembly 36 includes a reservoir actuator or force generating element, shown as, but not limited to, hydrogel 98, and a fluid distribution element, shown as, but not limited to, wick 100 in FIG. 6. Because FIG. 5 depicts delivery device 16 in the pre-activated position, hydrogel 98 is formed as a hydrogel disc and includes a concave upper surface 102 and a convex lower surface 104. As shown, wick 100 is positioned below hydrogel 98 and is shaped to generally conform to the convex shape of lower surface 104.

Substance delivery assembly 36 includes a microneedle activation element or microneedle actuator, shown as, but not limited to, torsion rod 106, and a latch element, shown as, but not limited to, latch bar 108. As explained in greater detail below, torsion rod 106 stores energy, which upon activation of delivery device 16, is transferred to one or more microneedles causing the microneedles to penetrate the skin. Substance delivery assembly 36 also includes a fluid reservoir plug 110 and plug disengagement bar 112. Bottom wall 61 is shown removed from base portion 32, and adhesive layer 22 is shown coupled to the lower surface of bottom wall 61. Bottom wall 61 includes one or more holes 114 that are sized and positioned to align with holes 28 in adhesive layer 22. In this manner, holes 114 in bottom wall 61 and holes 28 in adhesive layer 22 form channels, shown as needle channels 116.

As shown in FIG. 5, first support portion 62 includes a support wall 118 that includes a plurality of fluid channels 120. When assembled, wick 100 and hydrogel 98 are positioned on support wall 118 below drug reservoir 88. As shown, support wall 118 includes an upper concave surface that generally conforms to the convex lower surfaces of wick 100 and hydrogel 98. Fluid reservoir plug 110 includes a concave central portion 130 that is shaped to generally conform to the convex lower surface of support wall 118. First support portion 62 also includes a pair of channels 128 that receive the downwardly extending segments of torsion rod 106 such that the downwardly extending segments of torsion rod 106 bear against the upper surface of bottom wall 61 when delivery device 16 is assembled. Second support portion 63 includes a central cavity 122 that receives cup portion 94, U-shaped portion 96 and a portion of first portion 92 of drug channel arm 82. Second support portion 63 also includes a pair of horizontal support surfaces 124 that support latch bar 108 and a pair of channels 126 that slidably receive the vertically oriented portions of plug disengagement bar 112.

Referring to FIG. 6, a perspective, sectional view of delivery device 16 is shown attached or adhered to skin 132 of a subject prior to activation of the device. As shown, adhesive layer 22 provides for gross attachment of the device to skin 132 of the subject. Delivery device 16 includes a microneedle component, shown as, but not limited to, microneedle array 134, having a plurality of microneedles, shown as, but not limited to, hollow microneedles 142, extending from the lower surface of microneedle array 134. In the embodiment shown, microneedle array 134 includes an internal channel 141 allowing fluid communication from the upper surface of microneedle array 134 to the tips of hollow microneedles 142. Delivery device 16 also includes a valve component, shown as, but not limited to, check valve 136. Both microneedle array 134 and check valve 136 are mounted within cup portion 94. Drug channel 90 terminates in an aperture or hole 138 positioned above check valve 136. In the pre-activation or inactive position shown in FIG. 6, check valve 136 blocks hole 138 at the end of drug channel 90 preventing a substance, shown as, but not limited to, drug 146, within drug reservoir 88 from flowing into microneedle array 134. While the embodiments discussed herein relate to a drug delivery device that utilizes hollow microneedles, in other various embodiments, other microneedles, such as solid microneedles, may be utilized.

As shown in FIG. 6, in the pre-activation position, latch bar 108 is supported by horizontal support surfaces 124. Latch bar 108 in turn supports torsion rod 106 and holds torsion rod 106 in the torqued, energy storage position shown in FIG. 6. Torsion rod 106 includes a U-shaped contact portion 144 that bears against a portion of the upper surface of barrier film 86 located above cup portion 94. In another embodiment, U-shaped contact portion 144 is spaced above barrier film 86 (i.e., not in contact with barrier film 86) in the pre-activated position.

Delivery device 16 includes an activation fluid reservoir, shown as, but not limited to, fluid reservoir 147, that contains an activation fluid, shown as, but not limited to, water 148. In the embodiment shown, fluid reservoir 147 is positioned generally below hydrogel 98. In the pre-activation position of FIG. 6, fluid reservoir plug 110 acts as a plug to prevent water 148 from flowing from fluid reservoir 147 to hydrogel 98. In the embodiment show, reservoir plug 110 includes a generally horizontally positioned flange 150 that extends around the periphery of plug 110. Reservoir plug 110 also includes a sealing segment 152 that extends generally perpendicular to and vertically away from flange 150. Sealing segment 152 of plug 110 extends between and joins flange 150 with the concave central portion 130 of plug 110. The inner surface of base portion 32 includes a downwardly extending annular sealing segment 154. The outer surfaces of sealing segment 152 and/or a portion of flange 150 abut or engage the inner surface of annular sealing segment 154 to form a fluid-tight seal preventing water from flowing from fluid reservoir 147 to hydrogel 98 prior to device activation.

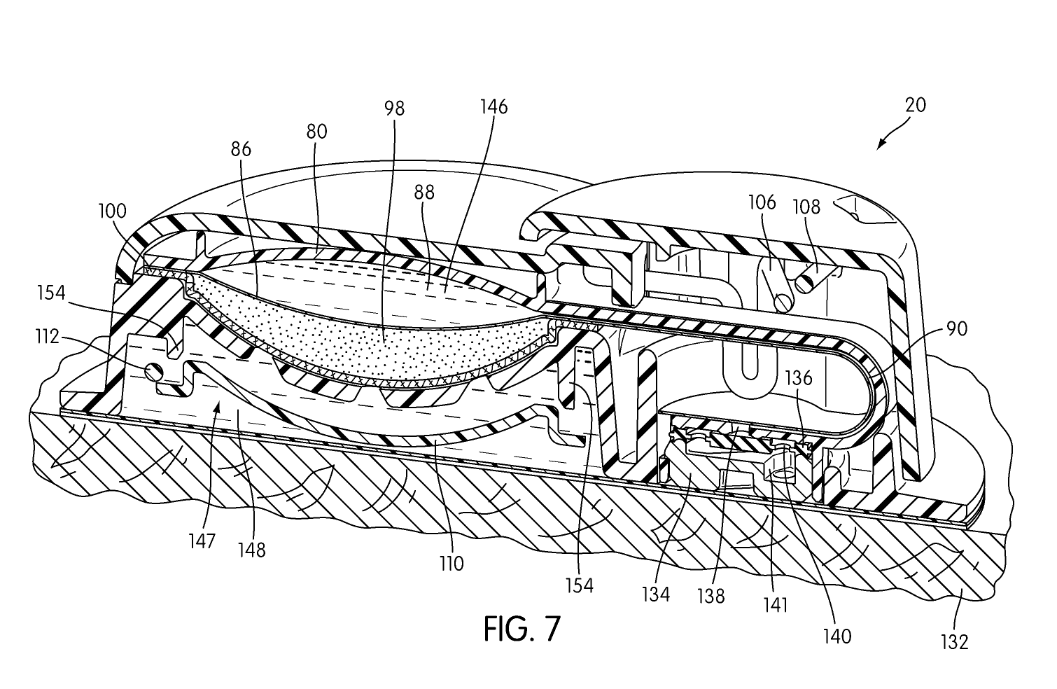

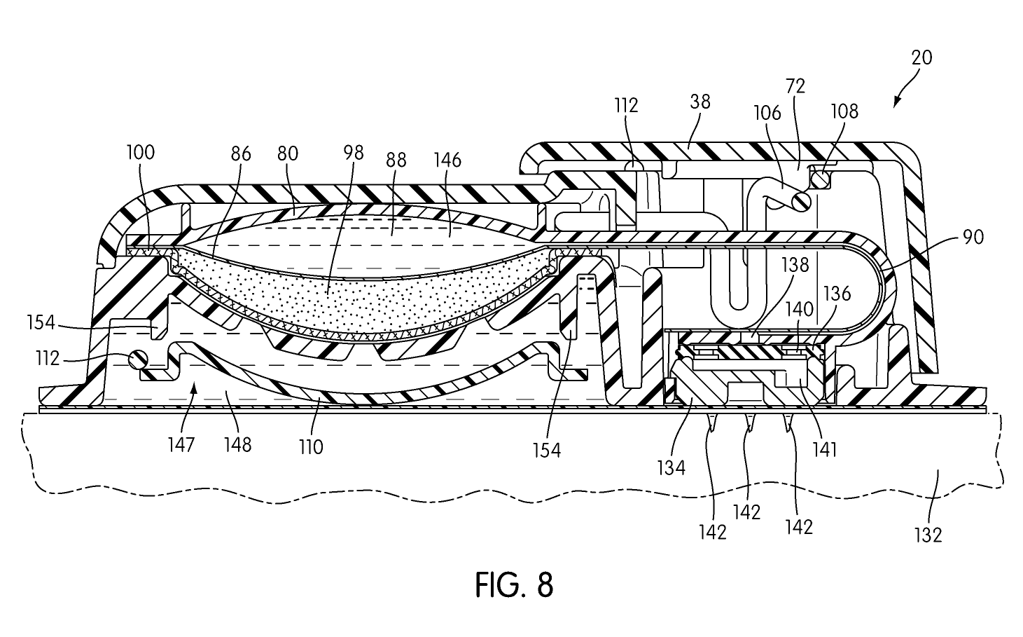

Referring to FIG. 7 and FIG. 8, delivery device 16 is shown immediately following activation. In FIG. 8, skin 132 is drawn in broken lines to show hollow microneedles 142 after insertion into the skin of the subject. To activate delivery device 16, button 20 is pressed in a downward direction (toward the skin). Movement of button 20 from the pre-activation position of FIG. 6 to the activated position causes activation of both microneedle array 134 and of hydrogel 98. Depressing button 20 causes first latch engagement element 72 and second latch engagement element 74 to engage latch bar 108 and to force latch bar 108 to move from beneath torsion rod 106 allowing torsion rod 106 to rotate from the torqued position of FIG. 6 to the seated position of FIG. 7. The rotation of torsion rod 106 drives microneedle array 134 downward and causes hollow microneedles 142 to pierce skin 132. In addition, depressing button 20 causes the lower surface of button top wall 38 to engage plug disengagement bar 112 forcing plug disengagement bar 112 to move downward. As plug disengagement bar 112 is moved downward, fluid reservoir plug 110 is moved downward breaking the seal between annular sealing segment 154 of base portion 32 and sealing segment 152 of reservoir plug 110.

With the seal broken, water 148 within reservoir 147 is put into fluid communication with hydrogel 98. As water 148 is absorbed by hydrogel 98, hydrogel 98 expands pushing barrier film 86 upward toward drug reservoir base 80. As barrier film 86 is pushed upward by the expansion of hydrogel 98, pressure within drug reservoir 88 and drug channel 90 increases. When the fluid pressure within drug reservoir 88 and drug channel 90 reaches a threshold, check valve 136 is forced open allowing drug 146 within drug reservoir 88 to flow through aperture 138 at the end of drug channel 90. As shown, check valve 136 includes a plurality of holes 140, and microneedle array 134 includes a plurality of hollow microneedles 142. Drug channel 90, hole 138, plurality of holes 140 of check valve 136, internal channel 141 of microneedle array 134 and hollow microneedles 142 define a fluid channel between drug reservoir 88 and the subject when check valve 136 is opened. Thus, drug 146 is delivered from reservoir 88 through drug channel 90 and out of the holes in the tips of hollow microneedles 142 to the skin of the subject by the pressure generated by the expansion of hydrogel 98.

In the embodiment shown, check valve 136 is a segment of flexible material (e.g., medical grade silicon) that flexes away from aperture 138 when the fluid pressure within drug channel 90 reaches a threshold placing drug channel 90 in fluid communication with hollow microneedles 142. In one embodiment, the pressure threshold needed to open check valve 136 is about 0.5-1.0 pounds per squire inch (psi). In various other embodiments, check valve 136 may be a rupture valve, a swing check valve, a ball check valve, or other type of valve the allows fluid to flow in one direction. In the embodiment shown, the microneedle actuator is a torsion rod 106 that stores energy for activation of the microneedle array until the activation control, shown as button 20, is pressed. In other embodiments, other energy storage or force generating components may be used to activate the microneedle component. For example, in various embodiments, the microneedle activation element may be a coiled compression spring or a leaf spring. In other embodiments, the microneedle component may be activated by a piston moved by compressed air or fluid. Further, in yet another embodiment, the microneedle activation element may be an electromechanical element, such as a motor, operative to push the microneedle component into the skin of the patient.

In the embodiment shown, the actuator that provides the pumping action for drug 146 is a hydrogel 98 that expands when allowed to absorb water 148. In other embodiments, hydrogel 98 may be an expandable substance that expands in response to other substances or to changes in condition (e.g., heating, cooling, pH, etc.). Further, the particular type of hydrogel utilized may be selected to control the delivery parameters. In various other embodiments, the actuator may be any other component suitable for generating pressure within a drug reservoir to pump a drug in the skin of a subject. In one exemplary embodiment, the actuator may be a spring or plurality of springs that when released push on barrier film 86 to generate the pumping action. In another embodiment, the actuator may be a manual pump (i.e., a user manually applies a force to generate the pumping action). In yet another embodiment, the actuator may be an electronic pump.

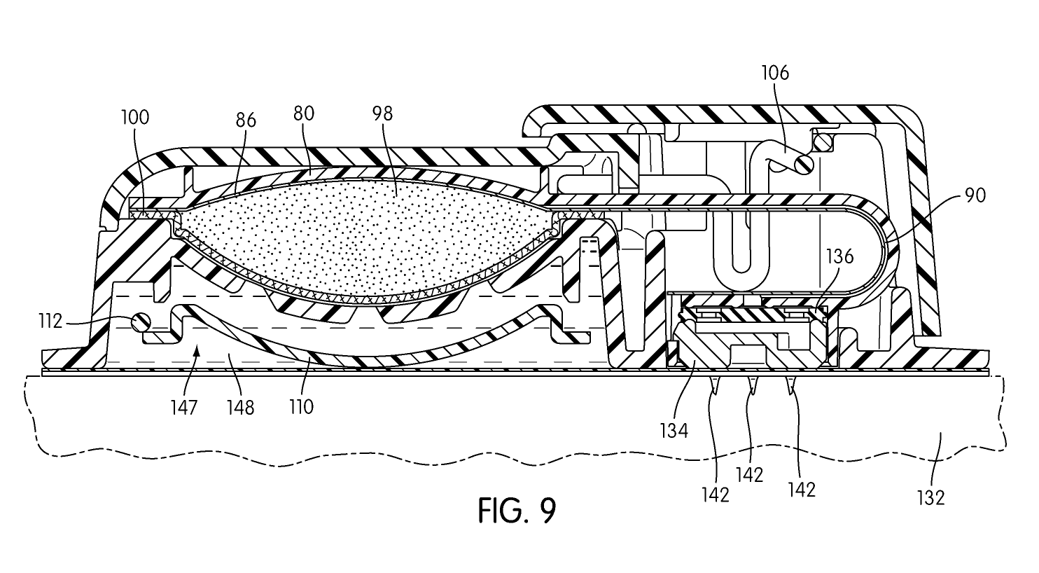

Referring to FIG. 9, delivery device 16 is shown following completion of delivery of drug 146 to the subject. In FIG. 9, skin 132 is drawn in broken lines. As shown in FIG. 9, hydrogel 98 expands until barrier film 86 is pressed against the lower surface of reservoir base 80. When hydrogel 98 has completed expansion, substantially all of drug 146 has been pushed from drug reservoir 88 into drug channel 90 and delivered to skin 132 of the subject. The volume of drug 146 remaining within delivery device 16 (i.e., the dead volume) following complete expansion by hydrogel 98 is minimized by configuring the shape of drug reservoir 88 to enable complete evacuation of the drug reservoir and by minimizing the volume of fluid pathway formed by drug channel 90, hole 138, plurality of holes 140 of check valve 136 and hollow microneedles 142. In the embodiment shown, delivery device 16 is a single-use, disposable device that is detached from skin 132 of the subject and is discarded when drug delivery is complete. However, in other embodiments, delivery device 16 may be reusable and is configured to be refilled with new drug, to have the hydrogel replaced, and/or to have the microneedles replaced.

In one embodiment, delivery device 16 and reservoir 88 are sized to deliver a dose of drug of up to approximately 500 microliters. In other embodiments, delivery device 16 and reservoir 88 are sized to allow delivery of other volumes of drug (e.g., up to 200 microliters, up to 400 microliters, up to 1 milliliter, etc.).

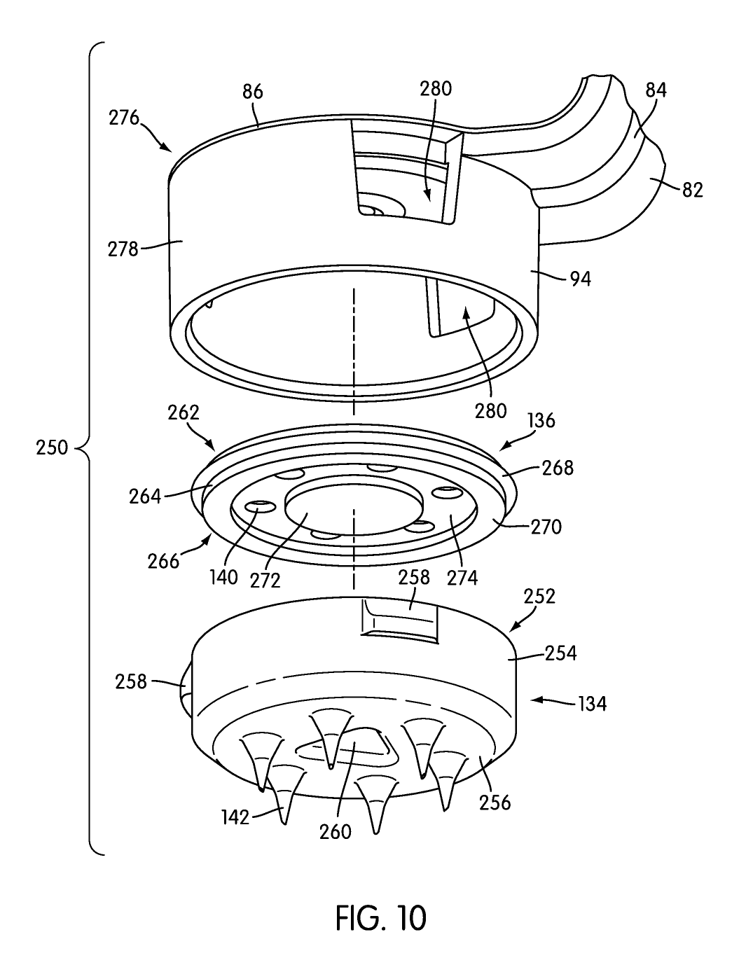

Referring generally to FIGS. 10-17, various embodiments of a microneedle component and a microneedle component assembly are shown. In the embodiments shown, components of the microneedle component assembly include features that facilitate assembly and handling during assembly. FIG. 10 shows a exploded perspective view of a microneedle component assembly 250 for a drug delivery device, such as delivery device 16, according to an exemplary embodiment. In the embodiment shown, microneedle component assembly includes a microneedle component, shown as microneedle array 134, a valve component, shown as check valve 136, and a microneedle attachment portion, shown as cup portion 94. As discussed above, cup portion 94 is coupled to channel arm 82 having groove 84.

In the embodiment shown in FIG. 10, microneedle array 134 includes an upper end 252 and a body portion. The body portion of microneedle array 134 includes a sidewall 254 and a bottom wall 256. Microneedle array 134 includes six microneedles 142 extending from and generally perpendicular to the outer surface of bottom wall 256. Microneedle array 134 also includes an engagement structure, shown as one or more tabs 258, to couple or attach microneedle array 134 to the microneedle attachment portion, shown as cup portion 94. Tabs 258 extend from the outer surface of sidewall 254 of microneedle array 134. Bottom wall 256 of microneedle array 134 includes a handling feature, shown as recess 260. In the embodiment of FIG. 10, microneedle array 134 is generally cylindrical having a generally circular cross-sectional area.

Check valve 136 includes an upper end 262, a sidewall 264, and a lower end 266. Check valve 136 includes a rim or bead 268 extending radially from sidewall 264. Check valve 136 includes a lower outer sealing portion 270, a lower inner portion 272 and a body wall 274, Check valve 136 includes six holes 140 that extend through body wall 274. Lower outer sealing portion 270 is shaped as a ring extending downward from the lower surface of body wall 274 near the periphery of check valve 136. Lower inner portion 272 is disc-shaped and extends downward generally from the center of the lower surface of body wall 274.

Cup portion 94 includes a top wall 276 and a sidewall 278 that extends downward from and generally perpendicular to the peripheral edge of top wall 276. As shown, barrier film 86 is adhered to the upper surface of top wall 276. Sidewall 278 includes one or more openings 280. To assemble microneedle component assembly 250, check valve 136 is placed into cup portion 94. Microneedle array 134 is placed into cup portion 94 below check valve 136 such that tabs 258 are received within openings 280 formed in the sidewall 278 of cup portion 94.

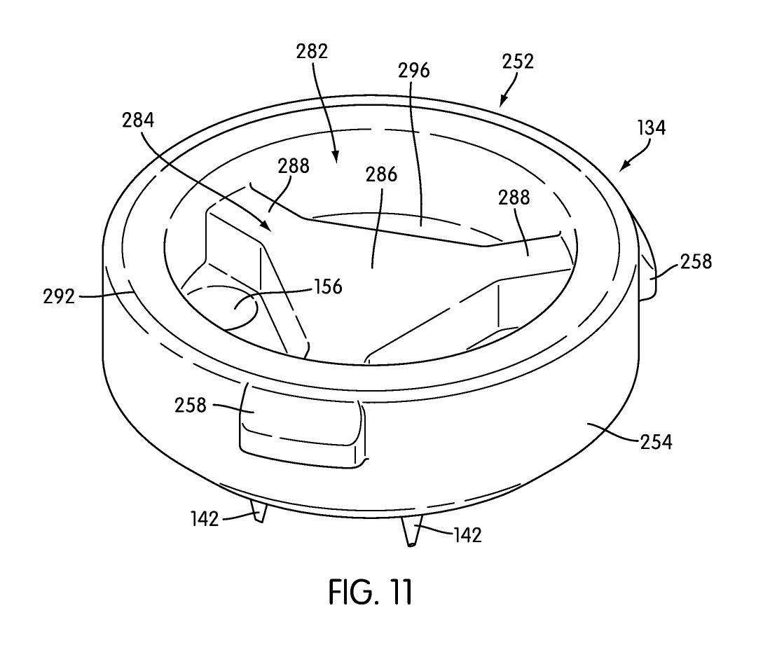

Referring to FIGS. 11-13, a microneedle component, shown as microneedle array 134, is depicted according to an exemplary embodiment. FIG. 11 is a perspective view from above of microneedle array 134. Microneedle array 134 includes a central recess 282. In the embodiment shown, recess 282 is defined by an inner surface of sidewall 254 and an upper surface of bottom wall 256. When microneedle array 134 is assembled within cup portion 94, recess 282 forms internal channel 141 (see FIG. 7) that provides fluid communication from upper end 252 of microneedle array 134 through microneedles 142. As shown in FIG. 11, microneedles 142 are cannulated, defining a central channel 156 that extends from the upper surface of bottom wall 256 through the tips of microneedles 142. This configuration places the tip of each microneedle 142 in fluid communication with internal channel 141 of microneedle array 134.

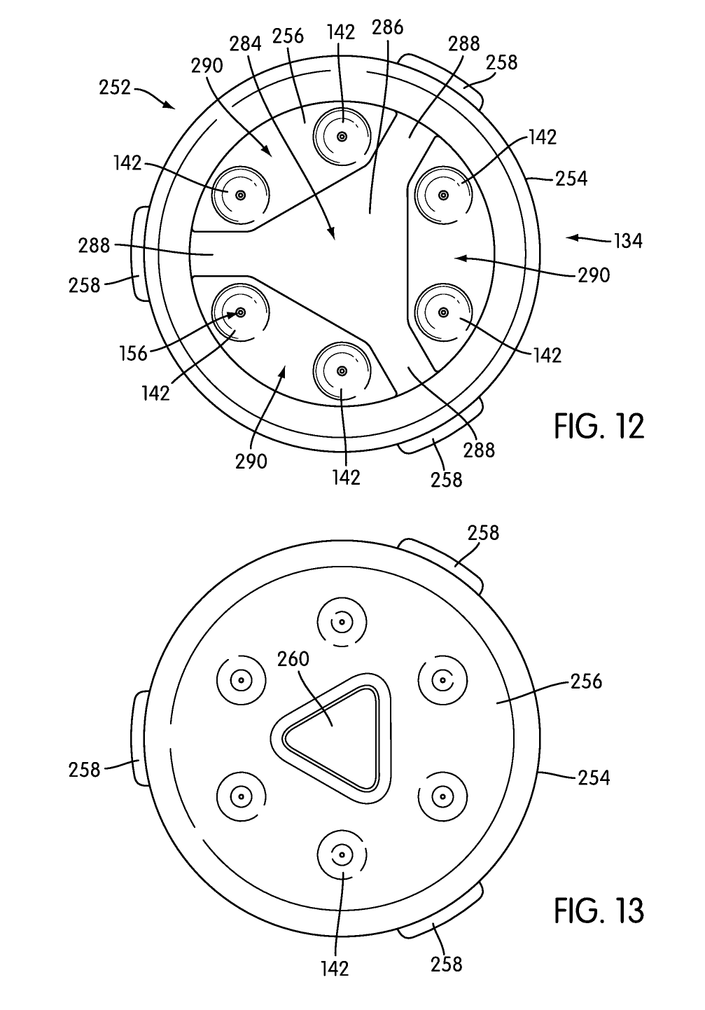

Microneedle array 134 includes a raised central section 284 located within recess 282. Raised central section 284 extends upward from the upper surface of bottom wall 256 partially filling recess 282. In the embodiment shown, raised section 284 includes a central triangular portion 286 and arm portions 288 extending from each corner of triangular portion 286 toward tabs 258. Raised section 284 acts to strengthen and support bottom wall 256 and sidewall 254 from loading that may occur during assembly or manufacture. As shown best in FIG. 12, raised section 284 divides recess 282 into three subsections 290, with each subsection 290 including two microneedles 142. As can be seen, each of the three subsections 290 have the same size and shape and the positioning of the two microneedles 142 in each subsection is the same. In this embodiment, raised section 284 reduces the volume of drug remaining within delivery device 16 (i.e., the dead volume) following complete expansion by hydrogel 98 (shown in FIG. 9) by decreasing the volume of recess 282.

In the embodiment shown in FIGS. 11-13, microneedle array 134 is generally cylindrical (i.e., has a generally circular cross-section) and includes three tabs 258 extending from the outer surface of sidewall 254. In the embodiment shown, tabs 258 are evenly spaced along the periphery of microneedle array 134 such that the center of each tab 258 is located approximately every 120 degrees. The even spacing of tabs 258 and the matching configuration of each subsection 290 is such that each 120 degree section of microneedle array 120 is the same as the other 120 degree sections of microneedle array 120. As will be discussed in more detail below, the 120 degree symmetry of microneedle array 134 facilitates assembly because the positioning of microneedles 142 relative to cup portion 94 following assembly does not depend on which tab 258 is received within which opening 280.

Referring to FIG. 11, the upper surface of sidewall 254 includes a sealing surface, shown as bead 292, extending from the upper surface of sidewall 254 of microneedle array 134. As explained in more detail below, bead 292 engages check valve 136 to form a seal when microneedle array 134 and check valve 136 are assembled within cup portion 94 (shown in FIG. 10). As shown in FIG. 13, microneedle array 134 includes a handling feature, shown as recess 260, formed in the lower surface of bottom wall 256. In the embodiment shown, recess 260 is generally triangular in shape with each corner of the triangle pointing toward one of tabs 258. In this embodiment, the triangular recess 260 is below and extends into triangular portion 286 of raised section 284. As explained in more detail below, recess 260, acts as a handling feature facilitating attachment and movement of microneedle array 134 during assembly. In other embodiments, recess 260 may be other non-circular or non-axisymmetric shapes to provide the alignment functionality discussed herein. In other embodiments, recess 260 may be circular or axisymmetric shapes with other structures or features (e.g., optical features, magnetic features, etc.) to ensure proper alignment during assembly.

In one embodiment, the components of microneedle array 134, including microneedles 142, sidewall 254, and bottom wall 256, are integrally formed from a plastic material by an injection molding process. In one embodiment, the components of microneedle array 134 are integrally formed by injection molding one of a variety of high-melt flow resins. In one embodiment, microneedle array 134 is made from liquid crystal polymer (LCP). Integrally forming microneedle array 134 of injection molded high-melt flow resin may be advantageous as this allows microneedles 142 to be integrally formed with sidewall 254 and bottom wall 256 of the microneedle component. The relatively large size of sidewall 254 and bottom wall 256 compared to the size of the integrally formed microneedles 142 provides a component that is large enough and durable enough to facilitate handling and attachment of microneedles 142. In one embodiment, microneedle array 134 may be made of a polymer reinforced with glass fiber. In another embodiment, microneedle array 134 may be made of a polymer that is not reinforced with glass fiber. In other embodiments, the microneedle component may be made via an embossing or etching process.

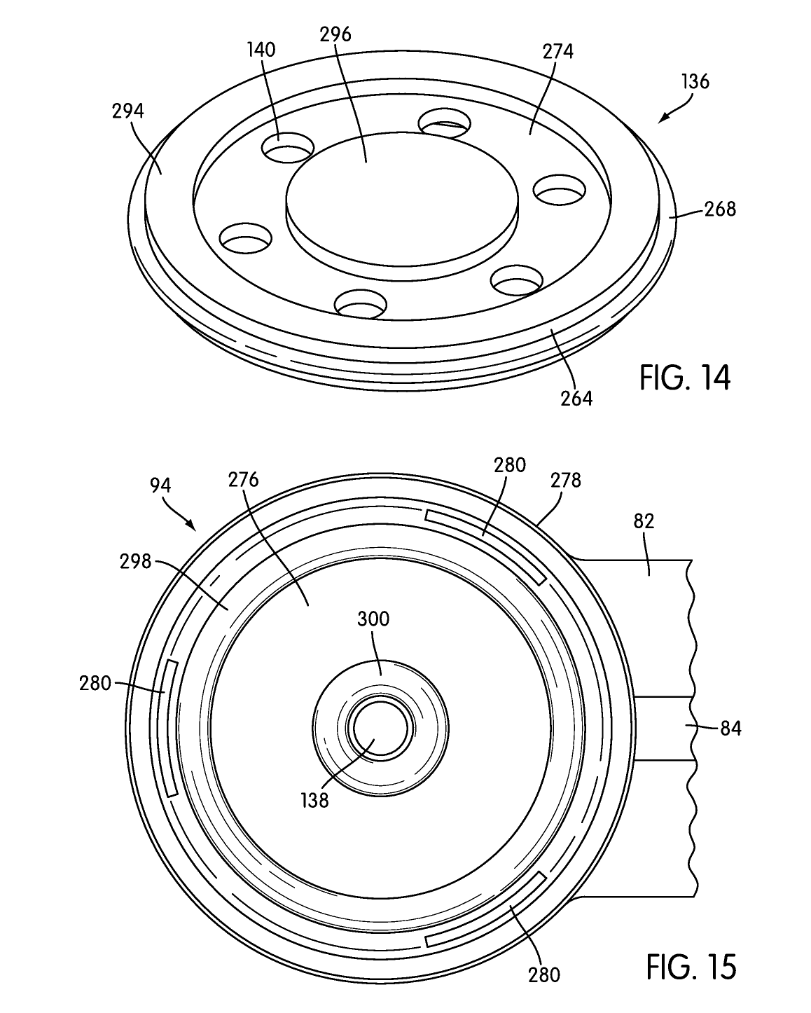

Referring to FIG. 14, a perspective view from above of a valve component, shown as check valve 136, is depicted in detail. Check valve 136 includes a rim or bead 268 extending radially from sidewall 264. Check valve 136 includes an upper outer sealing portion 294 and an upper inner sealing portion 296. Upper outer sealing portion 294 is shaped as a ring extending upward from the upper surface of body wall 274 near the periphery of check valve 136. Upper inner sealing portion 296 is disc-shaped and extends upward from generally the center of the upper surface of body wall 274. As shown in FIG. 14, holes 140 extend through the portion of body wall 274 that is located between upper outer sealing portion 294 and upper inner sealing portion 296. In this configuration, the portion of body wall 274 including holes 140 is recessed below the upper surfaces of upper outer sealing portion 294 and upper inner sealing portion 296. As explained in greater detail below, radial bead 268 and the sealing surfaces of check valve 136 provide for alignment of the components during assembly and provide a fluid tight seal after assembly.

FIG. 15 is a bottom view of cup portion 94 of drug channel arm 82 showing various structures within cup portion 94. Cup portion 94 includes a top wall 276 and a sidewall 278. Sidewall 278 defines three openings 280. Openings 280 are evenly spaced along sidewall 278 such that the center of each opening 280 is located approximately every 120 degrees. In this embodiment, the spacing of openings 280 matches the spacing of tabs 258 of microneedle array 134 (see FIG. 13). Cup portion 94 includes an outer sealing surface, shown as bead 298, and an inner sealing surface, shown as bead 300, that are ring-shaped and extend from the lower surface of top wall 276. As shown in FIG. 15, bead 298 is positioned near the inner surface of sidewall 278, and bead 300 encircles hole 138. As explained in greater detail below, beads 298 and 300 interact with check valve 136 to provide fluid tight seals after assembly.

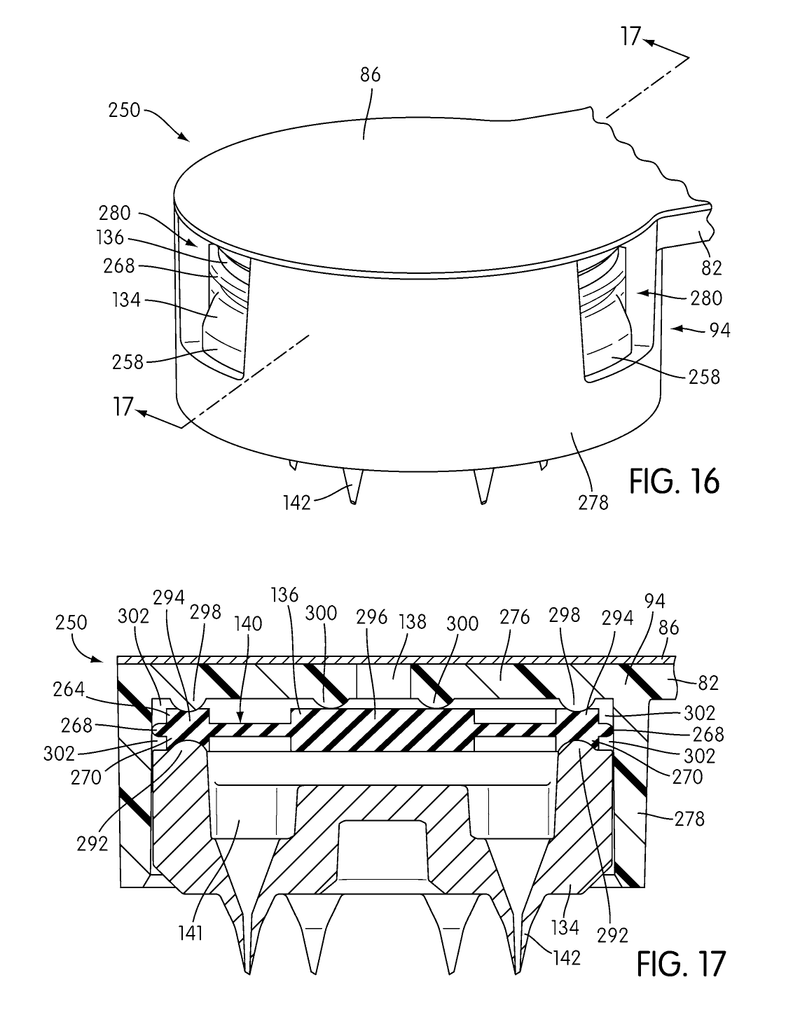

Referring to FIG. 16, microneedle component assembly 250 of drug delivery device 16 is depicted following assembly. As shown, check valve 136 is placed first into cup portion 94. Microneedle array 134 is then placed into cup portion 94 beneath check valve 136. When assembled, tabs 258 of microneedle array 134 extend through openings 280 of cup portion 94. In one embodiment, openings 280 are sized relative to tabs 258 to provide a snap-fit attachment between microneedle array 134 and cup portion 94. In one embodiment, check valve 136 is formed of a resilient material (e.g., silicone) that is compressed as microneedle array 134 is mounted within cup portion 94. In this embodiment, following assembly, the resilient material of check valve 136 expands pushing downward onto the upper surfaces of microneedle array 134. The downward force supplied by check valve 136 provides for a more stable fit between microneedle array 134 and cup portion 94 by forcing the lower surfaces of tabs 258 to engage the lower surfaces of openings 280 with greater force than if check valve 136 were not made from a resilient material.

While in the embodiment shown in FIG. 16, microneedle array 134 is mounted to cup portion 94 via a snap fit between tabs 258 and openings 280, microneedle array 134 may be mounted to cup portion 94 via other engagement structures. For example, in one embodiment, the engagement structure of microneedle array 134 may be a tapered sidewall allowing microneedle array 134 to be mounted within cup portion 94 via a press-fit taper lock between tapered sidewalls of microneedle array 134 and the sidewalls of cup portion 94. In another embodiment, the engagement structure of microneedle array 134 may be threads received within corresponding threads within cup portion 94. In another embodiment, the engagement structure may be an adhesive layer.

In one embodiment, microneedle array 134 is manipulated and mounted within cup portion 94 utilizing a tool attached to microneedle array 134. As shown in FIG. 13, microneedle array 134 includes a recess 260 that is configured to receive an engagement portion of an assembly tool. In this embodiment, the outer surface of the engagement portion of the tool engages the sidewalls of recess 260 to attach microneedle array 134 to the tool. With microneedle array 134 attached to the assembly tool, the assembly tool may be used to move microneedle array 134 into position to be assembled into cup portion 94. In the embodiment, shown, recess 260 is formed on the same surface of microneedle array 134 as microneedles 142. In this embodiment, because the handling feature, shown as recess 260, does not extend outwardly from the lower surface of bottom wall 256, recess 260 does not interfere with the insertion of microneedles 142 into the skin during activation. However, in other embodiments, the handling feature may extend from the outer surface of microneedle array 134.

In one embodiment, the engagement portion of the assembly tool may be a compressible portion that is press-fit within recess 260. In another embodiment, the engagement portion of the assembly tool may include expandable sections that expand to engage the sidewalls of recess 260. In yet another embodiment, recess 260 may include a magnetic material to assist in attachment to the assembly tool. In another embodiment, microneedle array 134 does not include a recess and the assembly tool includes a suction device that adheres to a surface of the microneedle array. In one embodiment, recess 260 acts as an alignment feature such that microneedle array 134 is aligned relative to the assembly tool in a predetermined manner. The engagement portion of the assembly tool may include a triangular keyed section configured to engage the triangular shape of recess 260 such that position of tabs 258 relative to the tool is known each time microneedle array 134 is manipulated by the tool. In another embodiment, recess 260 may include a notch or slot that receives a tab on the assembly tool such that microneedle array 134 is aligned relative to the assembly in a predetermined manner. The predetermined alignment of microneedle array 134 relative to the assembly tool facilitates alignment of tabs 258 with openings 280 of cup portion 94 during assembly (see FIG. 15).

In one embodiment, recess 260 allows for engagement with an assembly tool that is part of a robotic assembly device. In this embodiment, a robotic manipulation element, such as a robotic arm, may include the keyed engagement portion. In this embodiment, the predetermined alignment of microneedle array 134 relative to the assembly tool may be used to ensure alignment of tabs 258 with openings 280 as microneedle array 134 is mounted within cup portion 94. In this embodiment, the information related to the alignment of microneedle array 134 relative to the assembly tool may be one input to a control system controlling the robotic assembly device during coupling of microneedle array 134 to cup portion 94. The precise handling afforded by robotic handling of microneedle array 134 via recess 260 may be advantageous to limit inadvertent contact with and damage to microneedles 142 during manufacture of delivery device 16.

Referring to FIGS. 15 and 16, microneedle array 134 and cup portion 94 are configured to facilitate alignment of the parts during assembly. Because each 120 degree section of microneedle array 134 is the same (see FIGS. 12 and 13), the positioning of microneedles 142 relative to cup portion 94 does not depend on which tab 258 is received within which opening 280 during assembly. In other words, the positioning of microneedles 142 relative to cup portion 94 is the same without regard to which tab 258 is received within which opening 280. The alignment of microneedles 142 relative to cup portion 94 carries through to the assembly of drug delivery device 16 facilitating alignment of microneedles 142 with channels 116 formed in bottom wall 61 and adhesive layer 22 (see FIG. 5).

FIG. 17 shows a cross-section of microneedle component assembly 250 with microneedle array 134 and check valve 136 mounted within cup portion 94. As shown, check valve 136 is mounted above microneedle array 134 within cup portion 94. Bead 268 extending radially from sidewall 264 contacts the inner surface of sidewall 278 of cup portion 94. In this embodiment, because the diameter of check valve 136 through bead 268 is substantially the same as the internal diameter of cup portion 94, bead 268 ensures the axial center of check valve 136 is aligned with hole 138 following assembly. Further because check valve 136 is radially symmetrical, check valve 136 does not need to be rotationally aligned relative to cup portion 94 prior to assembly.

FIG. 17 shows the interaction between various sealing surfaces that results in the fluid tight seals within microneedle component assembly 250. Check valve 136 includes upper outer sealing portion 294 and lower outer sealing portion 270. Bead 298 of cup portion 94 engages upper outer sealing portion 294 and bead 292 of microneedle array 134 engages lower outer sealing portion 270. As shown in FIG. 17, lower outer sealing portion 270 deforms at the point of contact with bead 292, and upper outer sealing portion 294 may also deform at the point of contact with bead 298. As microneedle array 134 is mounted within cup portion 94, the material of check valve 136 is compressed forming seals between bead 298 and upper outer sealing portion 294 and between bead 292 and lower outer sealing portion 270. As shown in FIG. 17, the height of bead 268 is less than the height of check valve 136 through upper outer sealing portion 294 and lower outer sealing portion 270, resulting in open spaces 302 above and below bead 268.

As upper outer sealing portion 294 and lower outer sealing portion 270 are compressed during assembly, the material of the compressed sealing portions is able to move into the open spaces 302. Bead 268 provides for axial alignment of check valve 136 within cup portion 94, while also providing an open space to accommodate the compression and deformation of upper outer sealing portion 294 and lower outer sealing portion 270 created during assembly.

Prior to activation of hydrogel 98 (see FIG. 6), bead 300 engages upper inner sealing portion 296 of check valve 136. Following assembly, the material of check valve 136 is compressed onto bead 300 to form a fluid tight seal preventing drug from escaping through microneedle array 134 prior to device activation. As explained above, hole 138 positioned above upper inner sealing portion 296 is in fluid communication with drug reservoir 88. After activation of delivery device 16, fluid pressure increases in the region bounded by bead 300. When the fluid pressure reaches a threshold, upper inner sealing portion 296 flexes away from bead 300 breaking the seal. With the seal between bead 300 and upper inner sealing portion 296 broken, drug fluid from drug reservoir 88 is allowed to flow through holes 140 in check valve 136 into internal channel 141 of microneedle array 134 through the tips of microneedles 142.

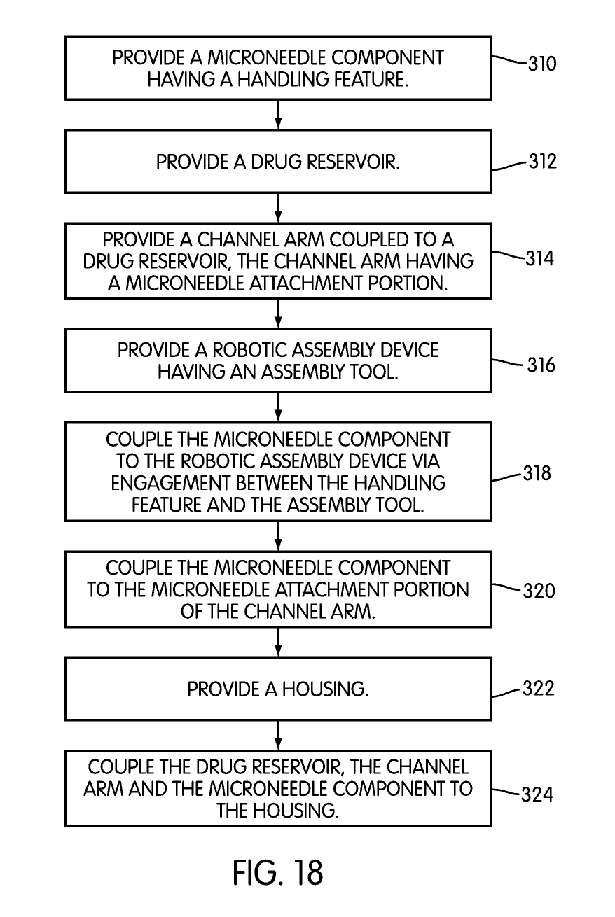

Referring to FIG. 18 a flow diagram of the assembly process for a microneedle drug delivery device is shown. At step 310, a microneedle component (e.g., microneedle array 134) having a handling feature (e.g., recess 260) is provided. At step 312, a drug reservoir (e.g., drug reservoir 88) is provided. At step 314, a conduit (e.g., channel arm 82) having a microneedle attachment portion (e.g., cup portion 94) is provided coupled to the drug reservoir. At step 316, a robotic assembly device having an assembly tool is provided. In one embodiment, the robotic assembly device is configured to manipulate the microneedle component to couple the microneedle component to the microneedle attachment portion of the conduit. In one embodiment, the robotic assembly device may be a part transfer robot manufactured by FANUC Robotics America, Inc.

At step 318, the microneedle component is coupled to the robotic assembly device via engagement between the handling feature and the assembly tool. In one embodiment, the handling feature acts as an alignment feature such that the microneedle component is aligned relative to the robotic assembly device in a predetermined manner after being coupled to the robotic assembly tool. In one embodiment, the tool includes an attachment portion that engages the inner surfaces of the sidewall of recess 260. At step 320, the microneedle component is coupled to the microneedle attachment portion via the robotic assembly device. In one embodiment, the robotic assembly device may position microneedle array 134 within cup portion 94 and may move (e.g., push) microneedle array 134 into cup portion 94 such that tabs 258 engage openings 280. As microneedle array 134 is pushed into engagement with cup portion 94, raised portion 284 (shown in FIG. 11) acts to strengthen the bottom wall and sidewall to resist or prevent plastic deformation that may otherwise result from the application of force to microneedle array 134 by the assembly tool. In one embodiment, the positioning of the microneedle component relative to the conduit and the coupling of the microneedle to the conduit via the robotic assembly device is based on the predetermined alignment of the microneedle component relative to the robotic assembly device. At step 322, a housing is provided, and at step 324, the assembled drug reservoir, channel arm, and microneedle component are coupled to the housing.

In one embodiment, the handling feature, shown as recess 260 (shown in FIG. 10), allows for robotic handling of microneedle array 134 during all steps of the manufacturing process. In this embodiment, the handling features enables the drug delivery device to be manufactured without the need for human contact with the microneedle component during any step of the assembly process. For example, in one embodiment, recess 260 of microneedle array 134 may be engaged by or coupled to a robotic tool located at the facility where microneedle array 134 is molded to remove the microneedle array from a molding device (e.g. an injection mold). With microneedle array 134 attached to the robotic tool, the robotic tool may then place microneedle array 134 into a container or packaging material to provide safe shipping and transport for the microneedle array prior to assembly with the drug delivery device. In this embodiment, molding of microneedle array 134 may occur at a facility or location that is different from the facility or location where assembly of microneedle array 134 with delivery device 16 occurs. When microneedle array 134 is to be attached to cup portion 94 of the drug delivery device (e.g., following transport of the packaged microneedle array 134 to the assembly facility), a robotic handling tool may be coupled to microneedle array 134 by engagement with recess 260 to remove microneedle array from the container or packaging, and as described above, microneedle array may be attached to cup portion 94 via the robotic handling tool. Thus, recess 260 may allow microneedle array to be robotically handled during all steps of the manufacturing, packaging, shipping and assembly processes.

Further modifications and alternative embodiments of various aspects of the invention will be apparent to those skilled in the art in view of this description. Accordingly, this description is to be construed as illustrative only. The construction and arrangements of the drug delivery device assembly and the drug delivery device, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

What is claimed is:

1. A device for delivering a drug to a subject, the device comprising:
a drug reservoir;
a conduit coupled to the drug reservoir; and
a microneedle component comprising:
a body;
an engagement structure coupling the microneedle component to the conduit;
a hollow microneedle extending from the body; and
a handling feature located on the body;
wherein the microneedle component is configured to be releasably coupled to an assembly tool via the handling feature during assembly of the device.

2. The device of claim 1, wherein the handling feature is configured such that the microneedle component is aligned relative to the assembly tool in a predetermined manner after coupling to the assembly tool.

3. The device of claim 1, wherein the handling feature includes a recess formed in the body of the microneedle component, and further wherein sidewalls of the recess are configured to be engaged by the assembly tool to couple the microneedle component to the assembly tool.

4. The device of claim 3, wherein the recess is non-circular.

5. The device of claim 4, wherein the recess is triangular, and further wherein the engagement structure includes a first tab, a second tab, and a third tab, and the conduit includes a first opening, a second opening, and a third opening, wherein the microneedle component is coupled to the conduit via engagement between the first tab and the first opening, engagement between the second tab and the second opening, and engagement between the third tab and the third opening.

6. The device of claim 5, wherein the engagement between the tabs and the openings is a snap-fit engagement.

7. The device of claim 5, wherein each corner of the triangular recess is aligned with one of the tabs.

8. The device of claim 7, wherein the body of the microneedle component includes a sidewall and has a generally circular cross-sectional area, and further wherein the tabs extend from the outer surface of the sidewall.

9. The device of claim 8, wherein the tabs are evenly spaced around the periphery of the sidewall.

10. A microneedle component of a drug delivery device, comprising:
a bottom wall having a lower surface;
a sidewall coupled to the bottom wall;
a microneedle extending from the lower surface of the bottom wall; and
a robotic handling feature formed in the lower surface of the bottom wall, the robotic handling feature configured to be releasably coupled to a robotic assembly tool during assembly of the drug delivery device.

11. The microneedle component of claim 10, wherein the robotic handling feature is configured such that the microneedle component is aligned relative to the robotic assembly tool in a predetermined manner after being coupled to the robotic assembly tool.

12. The microneedle component of claim 11, wherein the sidewall includes an inner surface and the bottom wall includes an upper surface, wherein the inner surface of the sidewall and the upper surface of the bottom wall define a central recess facing an upper end of the microneedle component, and further wherein the microneedle includes a central channel in fluid communication with the central recess.

13. The microneedle component of claim 12, wherein the robotic handling feature includes a recess formed in the lower surface of the bottom wall, the recess of the robotic handling feature facing a lower end of the microneedle component.

14. The microneedle component of claim 13, further comprising a plurality of tabs extending from an outer surface of the sidewall, the plurality of tabs configured to couple the microneedle component to the drug delivery device.

15. A method of manufacturing a drug delivery device, the method comprising:
providing a microneedle component having a robotic handling feature;
providing a drug reservoir;
providing a conduit coupled to the drug reservoir;
coupling the microneedle component to a robotic transfer device via engagement between the robotic handling feature and the robotic transfer device; and
coupling the microneedle component to the conduit with the robotic transfer device.

16. The method of claim 15, further comprising:
coupling the microneedle component to a second robotic transfer device via engagement between the robotic handling feature and the second robotic transfer device;
removing the microneedle component from a molding machine with the second robotic transfer device;
placing the microneedle component into a shipping container using the second robotic transfer device; and
removing the microneedle component from the shipping container with the robotic transfer device.

17. The method of claim 15, further comprising providing a housing and coupling the drug reservoir, conduit and microneedle component to the housing.

18. The method of claim 15, wherein the coupling the microneedle component step includes positioning the microneedle component within a portion of the conduit.

19. The method of claim 15, wherein the robotic handling feature is configured such that the microneedle component is aligned relative to the robotic transfer device in a predetermined manner after being coupled to the robotic transfer device.

20. The method of claim 19, wherein the coupling of the microneedle component to the conduit with the robotic transfer device is based on the predetermined alignment of the microneedle component relative to the robotic transfer device.
